(19) United States
(12) Patent Application Publication
(10) Pub. No.: US 2021/0186684 A1
(43) Pub. Date: Jun. 24, 2021

(54) THE MIDDLE EAR PROSTHESIS

(71) Applicants: UNIWERSYTET JAGIELLONSKI, Krakow (PL); AKADEMIA GORNICZO-HUTNICZA IM. STASZICA W KRAKOWIE, Krakow (PL)

(72) Inventors: Marcin KONIOR, Krakow (PL); Maciej KLACZYNSKI, Krakow (PL)

(21) Appl. No.: 16/499,569

(22) PCT Filed: Mar. 30, 2018
(86) PCT No.: PCT/PL2018/050014

§ 371 Date: Sep. 30, 2019

(30) Foreign Application Priority Data
Mar. 31, 2017 (PL) P.421075

(51) Int. Cl. A61F 2/18

(52) U.S. Cl. CPC A61F 2/18; A61F 2230/0006; A61F 2002/183

(57) ABSTRACT

The subject of the invention is a middle ear prosthesis (1), in particular of the middle ear conductive chain, comprising a first spring element (2) constituting a spring of at least one coil, having a length Lm in the range from 0.2 mm to 10 mm, and possibly a second spring element (3) constituting a spring of at least one coil, having a length Lₖ in the range from 0.1 mm to 4 mm, wherein N the first spring element (2) and the second spring element (3) are made of wire of a material having a Young's modulus E in the range from 7·10¹⁰ N/m² to 11.4·10¹⁰ N/m², a density p in the range from 4·10³ kg/m³ to 20·10³ kg/m³, a Poisson's ratio v in the range from 0.34 to 0.44, wherein the first spring element (2) is connected to the second spring element (3) in such a way that the rotational symmetry axis of the first spring element (2) is placed at an angle α₋₁ with respect to the rotational symmetry axis of the second spring element (3), included in the range from 5° to 160°.

The subject of the invention is a middle ear prosthesis, reconstructing the ossicular chain, used in middle ear surgery. The subject of the invention is applied, among others, in the surgical treatment of patients suffering from hearing disorders in the course of middle ear disorders, in particular with conductive hearing loss.

Hearing impairment constitutes the main problem of patients with middle ear disorders. Many of these cases require a surgical intervention. The multitude of prostheses types used in hearing improvement operations indicates the imperfection of the methods developed so far. A restriction of their effectiveness to selected requirements forces individuals performing such procedures to have many expensive models. So far, in the field of middle ear surgery, there have been no solutions, which enable matching one reconstruction system to different intraoperative situations. Therefore, It is necessary to create a prosthesis with wide adaptation possibilities, allowing for effective hearing improvement under various clinical conditions.

In the middle ear pathologies, during surgery, difficulties in reconstruction of the normal auditory pathway may arise. In particular, such a situation involves places where the surfaces of the modelled ossicles are in contact with each other or with the surface previously used to reconstruct prostheses. These are most often zones where prior to the beginning of the disease process articular connections were located, in which a change in the direction of forces and a change in the amount of transmitted acoustic energy took place.

Nowadays, an effective and useful ossicular prosthesis, which would be a basis for reconstructing the middle ear conductive chain, allowing for the aid by hearing improvement, which would be possible thanks to surgical treatment in patients suffering from hearing disorders in the course of middle ear disorders, is being sought. Such disorders include mostly chronic otitis media, however, the symptom of conductive hearing loss also occurs in the course of middle ear injuries, congenital defects, tympanosclerosis and middle ear neoplasms. Numerous patients, due to a difficult operating field, which includes the tympanic cavity and middle ear spaces, and because of the imperfections of the currently used ossicular prostheses, require further surgical procedures. Therefore, the search for better and better solutions becomes a necessity and a condition for the development in this field of therapeutic activity.

The Polish patent PL217562B1 discloses a chamber middle ear prosthesis for use in ear surgery to restore the function of the middle ear of a patient. The above-mentioned chamber middle ear prosthesis comprises a conical chamber formed by an annular wall having a shape of a truncated cone side wall whose wider base is closed by a membrane connected to a rigid element transmitting vibrations, and a narrower base is attached to the stapes base. The essence of the solution is that the opening in the narrower base is a capillary opening for filling the conical chamber with a liquid medium, especially a saline solution, and a flat annular disc with elements attaching the prosthesis to the stapes base is joined to the narrower base, the rigid element transmitting vibrations connected to the membrane being located outside the conical chamber.

In turn, the patent application P.412143 discloses a middle ear prosthesis of variable length. The essence of the middle ear prosthesis of variable length, having a holder fixing the prosthesis to the stapes and an element fixing the prosthesis to the tympanic membrane, is that it consists of S-shaped bars that are attached to the ends in the holder fixing the prosthesis to the stapes and the element fixing the prosthesis to the tympanic membrane. S-shaped bars, the prosthesis is built of, are connected to each other at the point of their contact with the use of stabilizing rings. In addition, the prosthesis is made of a shape memory material.

U.S. Pat. No. 6,277,148B1 discloses, among others, an attachment device for attaching the middle ear implant to structures in the middle ear. The attachment device consists of two loops connected with one another at a certain angle, made of wire, one loop being attached to the structures of the middle ear, e.g. to the ossicular chain system, and the other loop being intended to attach and maintain the implant part, e.g. in the form of a magnetic transducer. The above-mentioned system is only an element that fixes the active implant.

The technical problem addressing the present invention is to provide such a middle ear prosthesis that will effectively replace the ossicular function and thus allow for a reconstruction of the middle ear conductive chain. Furthermore, it is desirable that the middle ear prosthesis effectively transmits sound waves, preferably in the audible range, provides safety of use, provides functionality and intraoperative ease of modelling, and is also a universal structure allowing for the use under a variety of clinical conditions while maintaining a low manufacturing and application cost. Unexpectedly, the above-mentioned technical problems were resolved by the present invention.

The subject of the invention is a middle ear prosthesis, in particular of the middle ear conductive chain, characterized in that it comprises a first spring element constituting a spring of at least one coil, having a length Lm in the range from 0.2 mm to 10 mm, and possibly a second spring element constituting a spring of at least one coil, having a length Lₖ in the range from 0.1 mm to 4 mm, wherein the first spring element and the second spring element are made of wire of a material having a Young's modulus E in the range from 7·10¹⁰ N/m² to 11.4·10¹⁰ N/m², a density p in the range from 4·10³ kg/m³ to 20·10³ kg/m³, a Poisson's ratio v in the range from 0.34 to 0.44, wherein the first spring element is connected to the second spring element in such a way that the rotational symmetry axis of the first spring element is placed at an angle α₁ with respect to the rotational symmetry axis of the second spring element, included in the range from 5° to 160°.

In a preferred embodiment of the invention, the middle ear prosthesis comprises a third spring element attached to the second spring element at the end opposite to the first spring element, wherein the third spring element is a spring of at least one coil, having a length Ls in the range from 0.1 mm to 1 mm, made of wire of a material having a Young's modulus E in the range from 7·10¹⁰ N/m² to 11.4·10¹⁰ N/m², a density p in the range from 4·10³ kg/m³ to 20·10³ kg/m³, a Poisson's ratio v in the range from 0.34 to 0.44, wherein the third spring element is connected to the second spring element in such a way that the rotational symmetry axis of the third spring element is placed at an angle α₂ with respect to the rotational symmetry axis of the second spring element, included in the range from 5° to 160°.

In a further preferred embodiment of the invention, the spring elements form a continuous structure.

In a further preferred embodiment of the invention, the spring elements comprise a compression cylindrical spring, preferably with an outer diameter D_{z} in the range from 1.0 mm to 1.6 mm.

Preferably, the spring elements are made of wire of a circular cross section having a diameter d in the range from 0.1 mm to 0.3 mm.

Even more preferably, the spring elements have a spring pitch P in the range from 0.1 mm to 1.0 mm.

The middle ear prosthesis of the present invention is characterized by the versatility of use, functionality and intraoperative ease of modelling the final shape. Appropriate rigidity of the prosthesis affects the transfer of acoustic energy with its slight dispersion of it and the possibility of bending the spring, or changing the direction of forces. Proper elasticity allows for the transmission of waves along the axis of the prosthesis, but does not cause excessive energy absorption and the occurrence of a damping effect. The spring construction is characterized by the ability to be easily modelled by bending and twisting, which is extremely important in the spatial structure of the tympanic cavity. The construction of the spring also gives the possibility of easy connection with various anatomical parts of the ossicles to its ends or sides, effectively complementing the conductive chain. It should also be noted that despite the phenomenon of scarring of tissues during the healing process, which normally stiffens the transmission system, the coil structure of the prosthesis can at the same time affect the maintenance of a permanent mobility of the reconstructed system. Thanks to the effective transmission of the sound wave, the middle ear prosthesis of the present invention allows for a replacement of the ossicles function, thereby enabling a reconstruction of the middle ear conductive chain. Thanks to the lack of resonance at low frequencies, the prosthesis does not pose a threat to patients, which indicates the safety of its use. Moreover, the middle ear prosthesis is a simple structure made of a single material, which allows for the achievement of favourable economic factors.

Exemplary embodiments of the invention are presented in the figures of the drawings.

### BRIEF DESCRIPTION OF THE DRAWINGS

FIG. 1 is a front view of the middle ear prosthesis according to the first embodiment of the invention.

FIG. 2 is a partial cross-sectional front view of the first spring element of the solution from FIG. 1.

FIGS. 3A-3C show different variants of L-shaped middle ear prostheses in axonometric projection.

FIGS. 4A-4B show different variants of C-shaped middle ear prostheses in axonometric projection.

FIG. 5 is a photograph of an implanted middle ear prosthesis according to one embodiment of the present invention replacing a damaged anvil.

FIG. 6 is a photograph of an implanted middle ear prosthesis according to one embodiment of the present invention, stabilizing a disengaged incudostapedial joint.

FIG. 7 presents Tables 2 and 3.

### EXAMPLES

### Example 1

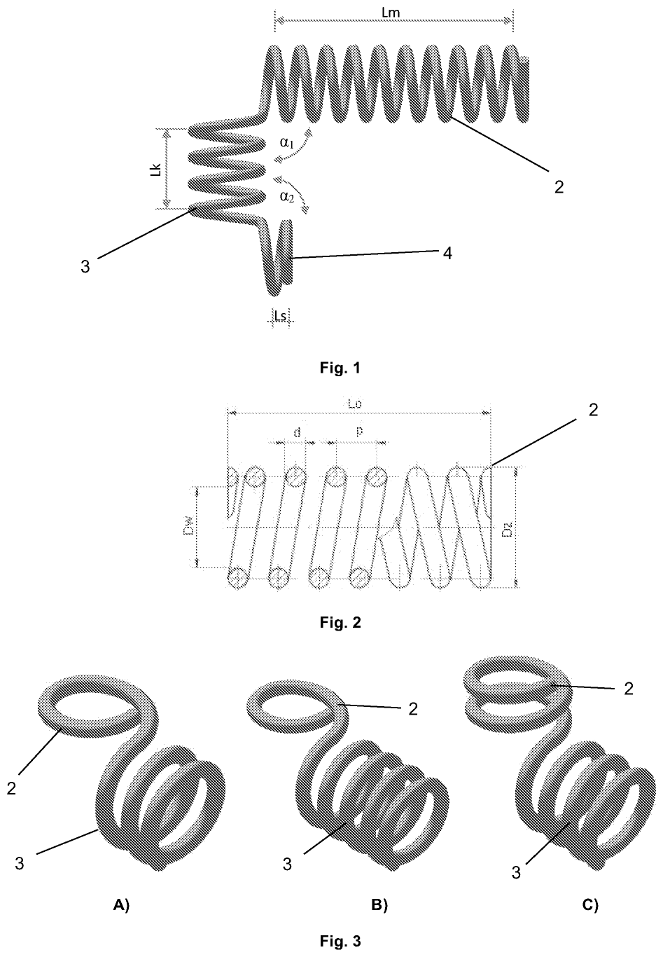

FIG. 1 is a schematic front view of the middle ear prosthesis 1 according to one possible embodiment of the present invention, while FIG. 2 is a partial cross-sectional front view of the first spring element 2 of the solution from FIG. 1. The middle ear prosthesis presented in FIG. 1 is a structure comprising three segments, made of one wire, constituting a continuous structure. The middle ear prosthesis 1 consists of the first spring element 2, which is a compression cylindrical spring having a length Lm in the range from 0.2 mm to 10 mm (depending on the needs). FIG. 2 indicates important structural parameters of the first spring element 2, also applicable to other spring elements 3, 4 of the discussed middle ear prosthesis 1. The second spring element 2, which is also a compression cylindrical spring having a length Lₖ in the range from 0.1 mm to 4 mm is connected at one end of the first spring element 2. The first spring element 2 is connected to the second spring element 3 in such a way that the rotational symmetry axis of the first spring element 2 is oriented at an angle α₁ with respect to the rotational symmetry axis of the second spring element 3, included in the range from 5° to 160°. Furthermore, the middle ear prosthesis presented in FIG. 1 comprises a third spring element 4 attached to the second spring element 3 at the end opposite to the first spring element 2. Similarly to the spring elements 2 and 3, the third spring element 4 is a compression cylindrical spring of at least one coil, having a length Ls in the range from 0.1 mm to 1 mm. Moreover, the third spring element 4 is connected to the second spring element 3 in such a way that the rotational symmetry axis of the third spring element 4 is placed at an angle α₂ with respect to the rotational symmetry axis of the second spring element 3, included in the range from 5° to 160°.

The whole middle ear prosthesis, or the spring elements 2, 3, 4 are made of wire of a circular cross section having a diameter d in the range from 0.1 mm to 0.3 mm. Each of the spring elements 2, 3, 4 has an outer diameter D_{z} in the range from 1.0 to 1.6 mm and a spring pitch P in the range from 0.1 mm to 1.0 mm. The wire used for the construction of the middle ear prosthesis is made of a material having a Young's modulus E in the range from 7·10¹⁰ N/m² to 11.4·10¹⁰ N/m², a density p in the range from 4·10³ kg/m³ to 20·10³ kg/m³, a Poisson's ratio v in the range from 0.34 to 0.44,

Individual embodiments of the presented middle ear prosthesis 1 will be shown in the following embodiments.

### Example 2

According to the present invention, ten variants of middle ear prostheses, divided into two groups: L-shaped (variants I-VI) and C-shaped (variants VII-X) have been made. Design parameters of these variants are summarized in Table 1.

TABLE 1A
Parameters of prostheses most often used in the implantation

| Variant | Cross | | | | | | | |
|---|---|---|---|---|---|---|---|---|
| of the | section | d | P | α₁ | α₂ | Lₘ | Lₖ | Lₛ |
| prosthesis | shape | [mm] | [mm] | [°] | [°] | [mm] | [mm] | [mm] |
| I | “L” | 1.35 | 0.4 | 90 | 0 | 0.4 | 0.8 | — |
| II | “L” | 1.35 | 0.4 | 90 | 0 | 0.4 | 1.6 | — |
| III | “L” | 1.5 | 0.5 | 90 | 0 | 0.5 | 1.0 | — |
| IV | “L” | 1.5 | 0.5 | 90 | 0 | 0.5 | 2.0 | — |
| V | “L” | 1.5 | 0.5 | 90 | 0 | 1.0 | 1.5 | — |
| VI | “L” | 1.35 | 0.4 | 90 | 0 | 0.8 | 1.2 | — |
| VII | “C” | 1.5 | 0.5 | 90 | 90 | 1.0 | 1.0 | 1.0 |
| VIII | “C” | 1.5 | 0.5 | 90 | 90 | 0.5 | 1.0 | 1.5 |
| IX | “C” | 1.35 | 0.4 | 90 | 90 | 0.8 | 0.8 | 0.8 |
| X | “C” | 1.35 | 0.4 | 90 | 90 | 0.4 | 0.8 | 1.2 |

TABLE 1B
Parameters of exemplary implanted prostheses
under clinical conditions

| Variant | Cross | | | | | | | |
|---|---|---|---|---|---|---|---|---|
| of the | section | d | P | α1 | α2 | Lm | Lk | Ls |
| prosthesis | shape | [mm] | [mm] | [°] | [°] | [mm] | [mm] | [mm] |
| A | “L” | 1.18 | 0.4 | 90 | 0 | 0.4 | 0.8 | — |
| B | “L” | 1.18 | 0.4 | 90 | 0 | 0.4 | 1.2 | — |
| C | “L” | 1.18 | 0.4 | 90 | 0 | 0.8 | 0.8 | — |
| D | “L” | 1.18 | 0.4 | 90 | 0 | 0.8 | 1.2 | — |
| E | “L” | 1.35 | 0.4 | 90 | 0 | 0.4 | 0.8 | — |
| F | “L” | 1.35 | 0.4 | 90 | 0 | 0.4 | 1.2 | — |
| G | “L” | 1.35 | 0.4 | 90 | 0 | 0.8 | 0.8 | — |
| H | “L” | 1.35 | 0.4 | 90 | 0 | 0.8 | 1.2 | — |
| I | “C” | 1.18 | 0.4 | 90 | 90 | 0.4 | 0.8 | 0.4 |
| J | “C” | 1.18 | 0.4 | 90 | 90 | 0.8 | 1.2 | 0.8 |
| K | “C” | 1.18 | 0.4 | 90 | 90 | 1.2 | 1.6 | 1.2 |
| L | “L” | 1.18 | 0.4 | 0 | 0 | 1.2 | 0 | — |
| M | “L” | 1.18 | 0.4 | 0 | 0 | 1.6 | 0 | — |
| N | “L” | 1.18 | 0.4 | 0 | 0 | 2.0 | 0 | — |
| O | “L” | 1.35 | 0.4 | 0 | 0 | 1.2 | 0 | — |
| P | “L” | 1.35 | 0.4 | 0 | 0 | 1.6 | 0 | — |
| R | “L” | 1.35 | 0.4 | 0 | 0 | 2.0 | 0 | — |
| S | “L” | 1.5 | 0.5 | 0 | 0 | 0.8 | 0 | — |
| T | “L” | 1.5 | 0.5 | 0 | 0 | 1.2 | 0 | — |
| U | “L” | 1.6 | 0.5 | 0 | 0 | 0.8 | 0 | — |
| W | “L” | 1.6 | 0.5 | 0 | 0 | 1.2 | 0 | — |

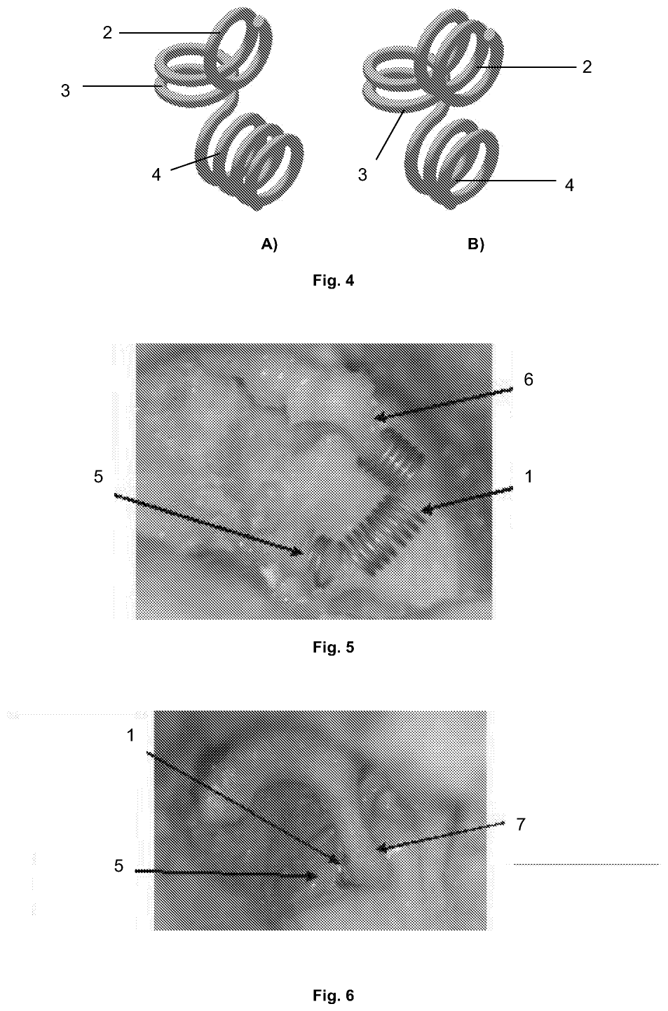

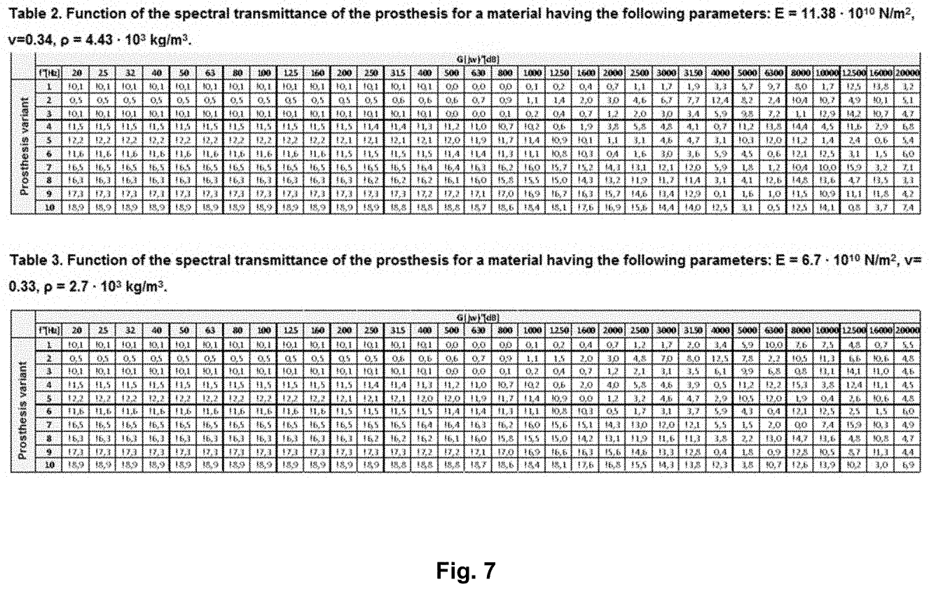

Some of the variants of the middle ear prostheses 1 are shown in the axonometric projections in the figures: variant I— FIG. 3A, variant II— FIG. 3B, variant VI—FIG. 3C, variant VII—FIG. 4B and variant VIII— FIG. 4A, respectively.

For the variants of the middle ear prosthesis presented in Table 1, the transmission of the acoustic wave, which can be described as a function of the spectral transmittance, i.e. the ratio of the input signal (acoustic wave) to the output signal as a frequency function, was determined according to the formula:

$$G ( j ω ) = \frac{Y ( j ω )}{X ( j ω )}$$

For the safe and proper operation of the middle ear prosthesis 1, the function G(jω) value for any frequency in the range 20 Hz+20 kHz should be in the range of ±20 dB. This range corresponds to a 10-fold amplification or attenuation of the acoustic signal in a linear scale. Particular attention should be paid to high attenuation values (greater than −10 dB) for the frequency band from the range of audiometry tests, or 125 Hz+8 kHz. This fact is associated with good transmission of the acoustic wave in the range of speech signal occurrence. The stimulation in the study was a forcing sinusoidal variable pressure level of 80 dB (corresponding to an acoustic pressure equal to 0.2 Pa).

Table 2 presents the function G(jω) value for the following variant of material parameters: E=11.38·10¹⁰ N/m², v=0.34, ρ=4.43·10³ kg/m³.

Table 3 presents the function G(jω) value for the following variant of material parameters: E=6.7·10¹⁰ N/m², v=0.33, ρ=2.7·10³ kg/m³.

Analysis of the obtained results indicates that the amplitudes do not reach critical values in the low frequency range (20 Hz-400 Hz). Therefore, the middle ear prosthesis 1 in the presented variants propagates well vibro-acoustic energy, without posing a threat of dangerous resonance in low frequencies.

### Example 3

The middle ear prosthesis 1 according to the present invention was examined in simulation tests on middle ear and auditory ossicles models. Simulations were performed on temporal bone models using an operating microscope, a surgical drive together with its equipment and adequate surgical instruments, so as to simulate the intraoperative conditions of the reconstruction of the middle ear transmission system the most precisely possible. In the trials, pathologies of individual auditory ossicles were simulated. The results showed the following indications for the application of the proposed prosthesis: PORP-type reconstruction, TORP-type reconstruction, incudostapedial anastomosis, anvil interposition, aid in solving various difficult and unusual situations (e.g. “empty cavity”).

FIG. 5 is a photograph of an implanted middle ear prosthesis 1 according to one embodiment of the present invention replacing a damaged anvil, whereas FIG. 6 is a photograph of an implanted middle ear prosthesis 1 according to one embodiment of the present invention, stabilizing a disengaged incudostapedial joint. In the example presented in FIG. 5 the middle ear prosthesis 1, and in fact the first spring element 2, was attached to the head of the stapes 5, while on the other side, the third spring element 4 was attached to the manubrium of the hammer 6. In this case, the middle ear prosthesis 1 served as an acoustic transducer ensuring the reconstruction of the middle ear conductive chain. On the other hand, in the example presented in FIG. 5 the middle ear prosthesis 1 was attached between the head of the stapes 5 and the long crus of the anvil 7. In this case, the usefulness of the middle ear prosthesis 1 according to the present invention as an annular stabilizer of the incudostapedial joint was demonstrated.

The methodology for the use of middle ear prosthesis 1 according to the present invention consisted in the appropriate selection of the correct length and shape, changing the obtained length by cutting with a surgical knife or scissors of a relevant fragment of the produced element and/or changing the form made by the operator using microsurgical needles bending the prosthesis 1 to obtain a required shape.

### Example 4 Clinical Tests

The prostheses according to the invention have been used in the surgical treatment of middle ear disorders requiring a reconstruction of the ossicular chain.

The results of observations of patients qualified for the clinical trial “UNISPRING—a new system of modelled microprostheses of auditory ossicles reconstructing the middle ear conductive chain”.

In the group participating in the clinical trial, different variants of the prostheses according to the invention were implanted in 13 patients divided into two groups: 7 people and 6 people.

The second group of patients has not completed the full observation period yet, but the results are so far encouraging. The results presented below originate from the analysis of data collected during the observation of patients from the first group.

| | | | Manufacture |
|---|---|---|---|
| Type of surgery | Description | Prosthesis | method |
| INCUDO- | incudostapedial | UNISPRING | L: |
| STAPEDOPEXIA | anastomosis | 1.18 | short segment: 1-2 |
| | | UNISPRING | coils |
| | | 1.35 | long segment: 2-3 |
| | | | coils |
| MALEO- | maleotapedial | UNISPRING | C: |
| STAPEDOPEXIA | anastomosis | 1.18 | 1. arm: 1-3 coils |
| | | UNISPRING | central arm: 2-4 |
| | | 1.35 | coils |
| | | | 3. arm: 1-3 coils |
| PORP | partial ossicular | UNISPRING | straight segment of |
| | replacement | 1.50 | the length: 2-3 coils |
| | prosthesis | UNISPRING | |
| | | 1.60 | |
| TORP | total ossicular | UNISPRING | straight segment of |
| | replacement | 1.18 | the length: 3-5 coils |
| | prosthesis | UNISPRING | |
| | | 1.35 | |

The prosthesis according to the invention has proved to be a safe, effective, functional and ergonomic solution intended for the surgical treatment of the ossicular conductive chain.

The table below presents the scope of applications for which the prosthesis according to the invention was used, allowing for individual adjustment to the anatomical conditions of the patient and the type of pathology.

### Designation of Prosthesis Type:

| Prosthesis | |
|---|---|
| designation | Description |
| UNISPRING | A cylindrical spring with an outer diameter of 1.18 mm, |
| 1.18 | and length and shape adapted to anatomical conditions |
| UNISPRING | A cylindrical spring with an outer diameter of 1.35 mm, |
| 1.35 | and length and shape adapted to anatomical conditions |
| UNISPRING | A cylindrical spring with an outer diameter of 1.50 mm, |
| 1.50 | and length and shape adapted to anatomical conditions |
| UNISPRING | A cylindrical spring with an outer diameter of 1.60 mm, |
| 1.60 | and length and shape adapted to anatomical conditions |

Detailed evaluation of the effectiveness of the so far collected material for the whole I group has been made based on the analysis of the results of the pure tone audiometry, speech audiometry and a subjective assessment and it is presented below:

### A.:

Pure tone audiometry—mean values of pre-operative reserve and improvement (reduction in the post-operative reserve by the following values):

| | | Average improvement by: |
|---|---|---|
| | The value of the auditory reserve | [dB] |
| frequency | before the procedure | 12 months after |
| [Hz] | [dB] | the procedure |
| 125 | 39 | 18 |
| 250 | 44 | 35 |
| 500 | 39 | 25 |
| 1000 | 27 | 20 |
| 2000 | 16 | 7 |
| 4000 | 15 | 15 |
| 8000 | 5 | 0 |

An improvement of the results within the described spectrum, including the so-called “closing reserve” phenomenon (value of the auditory reserve below 10 dB) for frequencies relevant to speech reception was obtained—for the following frequencies: 250 Hz, 500 Hz, 1 000 Hz, 2 000 Hz, 4 000 Hz.

### B.:

Speech audiometry—mean values:

| loudness level | Understanding [%] | |
|---|---|---|
| [dB] | Before the procedure | 12 months after the procedure |
| 10 | 0 | 0 |
| 20 | 0 | 0 |
| 30 | 0 | 0 |
| 40 | 0 | 0 |
| 50 | 0 | 2 |
| 60 | 7 | 2 |
| 70 | 40 | 72 |
| 80 | 69 | 87 |
| 90 | 85 | 87 |
| 100 | 87 | 87 |

A significant improvement in the threshold of understanding was achieved—a reduction in the loudness level by 20 dB (from 60 to 40 dB). The maximum capacity of using the auditory capabilities (90-100% of test understanding) in the loudness range of 60-80 dB was achieved in all operated patients. Before the procedure, a similar situation occurred in 1 patient (100% understanding at 60 dB).

### C.:

Average value of subjective assessment:

Average value of subjective assessment [points]

| Before the procedure | 12 months after the procedure |
|---|---|
| 2.6 | 8.7 |

In all patients, an improvement of hearing assessed subjectively in the scale of 0-10 points was achieved.

1. A middle ear prosthesis (1), in particular of the middle ear conductive chain, characterized in that it comprises a first spring element (2) constituting a spring of at least one coil, having a length Lm in the range from 0.2 mm to 10 mm, and possibly a second spring element (3) constituting a spring of at least one coil, having a length Lₖ in the range from 0.1 mm to 4 mm, wherein the first spring element (2) and the second spring element (3) are made of wire of a material having a Young's modulus E in the range from 7·10¹⁰ N/m² to 11.4·10¹⁰ N/m², a density p in the range from 4·10³ kg/m³ to 20·10³ kg/m³, a Poisson's ratio v in the range from 0.34 to 0.44, wherein the first spring element (2) is connected to the second spring element (3) in such a way that the rotational symmetry axis of the first spring element (2) is placed at an angle α₁ with respect to the rotational symmetry axis of the second spring element (3), included in the range from 5° to 160°.

2. The middle ear prosthesis (1) according to claim 1, characterized in that it comprises a third spring element (4) attached to the second spring element (3) at the end opposite to the first spring element (2), wherein the third spring element (4) is a spring of at least one coil, having a length Ls in the range from 0.1 mm to 1 mm, made of wire of a material having a Young's modulus E in the range 7·10¹⁰ N/m² to 11.4·10¹⁰ N/m², a density p in the range from 4·10³ kg/m³ to 20·10³ kg/m³, a Poisson's ratio v in the range from 0.34 to 0.44, wherein the third spring element (4) is connected to the second spring element (3) in such a way that the rotational symmetry axis of the third spring element (4) is placed at an angle α₂ with respect to the rotational symmetry axis of the second spring element (3), included in the range from 5° to 160°.

3. The middle ear prosthesis (1) according to claim 1, characterized in that the spring elements (2, 3, 4) form a continuous structure.

4. The middle ear prosthesis (1) according to claim 1, characterized in that the spring elements (2, 3, 4) form a compression cylindrical spring, preferably with an outer diameter D_{z} in the range from 1.0 mm to 1.6 mm.

5. The middle ear prosthesis (1) according to claim 1, characterized in that the spring elements (2, 3, 4) are made of wire of a circular cross section having a diameter d in the range from 0.1 mm to 0.3 mm.

6. The middle ear prosthesis (1) according to claim 1, characterized in that the spring elements (2, 3, 4) have a spring pitch P in the range from 0.1 mm to 1.0 mm.
